(19) United States
(12) Patent Application Publication
(10) Pub. No.: US 2021/0019834 A1
(43) Pub. Date: Jan. 21, 2021

(54) METHOD AND SYSTEM FOR PROCESSING INSURANCE CLAIMS

(71) Applicant: iNube Software Solutions Pvt. Ltd, Bengaluru (KR)

(72) Inventors: RAMPRASAD SANJEEVI, Bengaluru (IN); SADISHKUMAR VENKATESAN, Bengaluru (IN); DAKSHINA MOORTHY JONNALAGADDA, Bengaluru (IN)

(21) Appl. No.: 16/879,989

(22) Filed: May 21, 2020

(30) Foreign Application Priority Data
Jul. 17, 2019 (IN) 201941028772

(51) Int. Cl. G06Q 40/08; G06Q 10/10; G16H 70/20; G06Q 10/00; G16H 10/60; G06F 16/28

(52) U.S. Cl. CPC G06Q 40/08; G06Q 10/107; G06F 16/285; G06Q 10/20; G16H 10/60; G16H 70/20

(57) ABSTRACT

A method and a system for processing an insurance claim payable to a user by an insurance company is disclosed. In an embodiment, the method may include receiving a user identity and a user request for awarding of an insurance claim, and classifying the user request in one of a plurality of pre-defined categories. The method may further include correlating the user request with a digital data matrix. The method may further include retrieving one or more parameters associated with the user identity, wherein the one or more parameters associated with the user identity are stored in the data repository. The method may further include; and determining an insurance claim amount payable to the user by the insurance company, based on the correlation with the data matrix and the one or more parameters associated with the user identity and an insurance policy.

### CROSS-REFERENCE TO RELATED APPLICATIONS

This application claims priority benefits to Indian Patent Application No. 201941028772, filed on Jul. 17, 2019, which is hereby incorporated by reference in its entirety.

### TECHNICAL FIELD

This disclosure relates generally to insurance claims, and more particularly to a method and a system for processing an insurance claim or a treatment cost, payable to a user by an insurance company or a third party administrator (TPA), or under a self-funded or a sponsored medical scheme.

### BACKGROUND

Insurance play an important role in protecting buyers of insurance policies from financial loss. Various kinds of insurance policies may be offered by insurance companies (insurers), that may include health insurance, automobile insurance, etc. The buyers may seek insurance claims, in view of an incurred or impending financial expense. As it will be appreciated by those skilled in the art, the financial expense may be towards treatment of a health condition (for example, disease or physical injury), repairing or maintenance of an automobile, etc. Insurance claim processing, within the purview of Insurance Policy terms and conditions, plays an important part in adjudicating an amount of the insurance claims being sought by the buyer.

Insurance claims processing may involve a number of steps, including receiving a request from a buyer for awarding insurance claims payable by an insurance company, adjudicating an amount payable to the buyer, providing updates to the buyer on the status of claim processing, and so on. As it will be appreciated, most of these steps involve significant manual intervention. The manual intervention may not only make the overall process of claim processing slower, but also prone to errors.

### SUMMARY

In one embodiment, a method of processing an insurance claim payable to a user by an insurance company. The method may include receiving a user identity and a user request for awarding of insurance claims payable by the insurance company. The method may further include classifying the user request in one of a plurality of pre-defined categories. The method may further include correlating the user request with a digital data matrix, wherein the digital data matrix is stored in a data repository. The method may further include retrieving one or more parameters associated with the user identity, wherein the one or more parameters associated with the user identity are stored in the data repository. The method may further include determining an insurance claim amount payable to the user by the insurance company, based on the correlation with the data matrix and the one or more parameters associated with the user identity and an insurance policy.

In another embodiment, a system for processing an insurance claim payable to a user by an insurance company. The system may include a claim processing device. The claim processing device may further include a processor, and a memory communicatively coupled to the processor. The memory stores processor instructions, which, on execution, may cause the processor to receive a user identity and a user request for awarding of insurance claims payable by the insurance company. The processor instructions, on execution, may further cause the processor to classify the user request in one of a plurality of pre-defined categories, and correlate the user request with a digital data matrix, wherein the digital data matrix is stored in a data repository. The processor instructions, on execution, may further cause the processor to retrieve one or more parameters associated with the user identity, wherein the one or more parameters associated with the user identity are stored in the data repository. The processor instructions, on execution, may further cause the processor to determine an insurance claim amount payable to the user by the insurance company based on the correlation with the data matrix and the one or more parameters associated with the user identity, and an insurance policy.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles.

FIG. 1 is a block diagram illustrating a system for processing insurance claims, in accordance with an embodiment.

FIG. 2 illustrates a block diagram of a memory of a claim processing device for processing insurance claims, in accordance with an embodiment.

FIG. 3 illustrates a block diagram of a claim processing device for processing insurance claims, in accordance with an embodiment.

FIG. 4 illustrates a flowchart of a method for processing insurance claims, in accordance with an embodiment.

FIG. 5 illustrates an exemplary process for providing a centralized view of underwriting rules and pricing guidelines for processing an insurance claim, in accordance with an embodiment

### DETAILED DESCRIPTION

Exemplary embodiments are described with reference to the accompanying drawings. Wherever convenient, the same reference numbers are used throughout the drawings to refer to the same or like parts. While examples and features of disclosed principles are described herein, modifications, adaptations, and other implementations are possible without departing from the spirit and scope of the disclosed embodiments. It is intended that the following detailed description be considered as exemplary only, with the true scope and spirit being indicated by the following claims. Additional illustrative embodiments are listed below.

Glossary of Abbreviations

| | | |
|---|---|---|
| 1 | TPA | Third Party Administrator |
| 2 | ACORD | Association for Cooperative Operations Research and Development |
| 3 | WLAN | Wireless Local Area Network |
| 4 | LTE | Long Term Evolution |
| 5 | WiMAX | Worldwide Interoperability for Microwave Access |
| 6 | GPRS | General Packet Radio Service |
| 7 | ROM | Read Only Memory |
| 8 | PROM | Programmable ROM |
| 9 | EPROM | Erasable PROM |
| 10 | EEPROM | Electrically EPROM |
| 11 | DRAM | Dynamic Random Access Memory |
| 12 | SRAM | Static Random-Access memory |
| 13 | TAT | Turn-Around-Time |
| 14 | UHID | Unique Health Insurance Identification Number |
| 15 | COI | Certificate of Insurance |
| 16 | ICD | International Classification of Diseases |
| 17 | PCS | Procedure Coding System |
| 18 | CPT | Current Procedures Terminology |
| 19 | SNOWMED-CT | Systematized Nomenclature of Medicine-Clinical Terms |
| 20 | RTO | Regional Transport Office |
| 21 | SoC | Schedule Of Charges |
| 22 | CHD | Coronary Heart Disease |
| 23 | CABG | Coronary Artery Bypass Grafting |
| 24 | OT | Operation Theatre |
| 25 | ICU | Intensive Care Unit |
| 26 | AI | Artificial Intelligence |
| 27 | CNN | Convolutional Neural Network |
| 28 | LDAP | Lightweight Directory Access Protocol |
| 29 | LIFO | Last-in, first-out |
| 30 | FIFO | First-in, first-out |
| 31 | API | Application Program Interface |
| 32 | PPN | Preferred Provider Network |
| 33 | ROHINI | Registry of Hospitals in Network of Insurance |
| 34 | IIB | Insurance Information Bureau (India) |
| 35 | MSCRM | Microsoft Dynamics Customer Relationship Management |
| 36 | CRM | Customer Relation Management |
| 37 | LOS | Length of Stay |
| 38 | OPD | Out Patient Department |
| 39 | SPOC | Single Point Of Contact |
| 40 | MoU | Memorandum Of Understanding |
| 41 | RDLC | Report Definition Language Client Side |
| 42 | DNF | Data Not Found |
| 43 | DMS | Document Management System |
| 44 | CIMS | Content Management Interoperability Services |

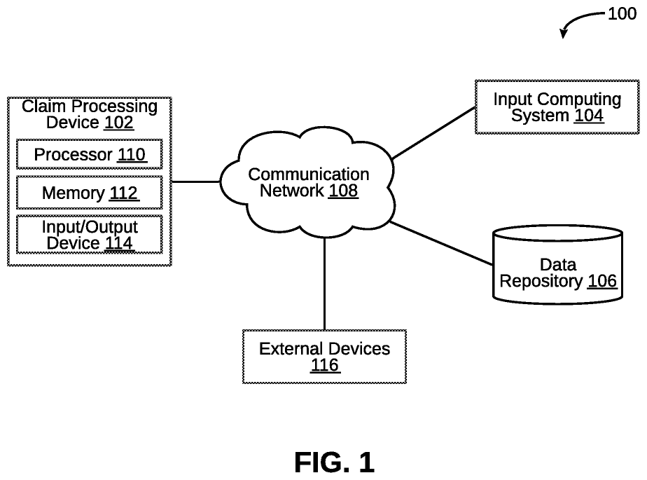

In one embodiment, a system 100 for processing an insurance claim or a treatment cost payable to a user by an insurance company or a TPA, or under a self-funded or a sponsored medical scheme is illustrated in the FIG. 1, in accordance with an embodiment. The system 100 may include a claim processing device 102, an input computing system 104, and a data repository 106. The claim processing device 102 may be a computing device having capability of processing insurance claims. Examples of the claim processing device 102 may include, but are not limited to, server, desktop, laptop, notebook, netbook, tablet, smartphone, mobile phone, application server, sever, or the like. It may be noted that the system 100 may perform various functions which are in compliance with one or more standards set by one or more global standards-setting bodies. For example, the one or more global standards-setting bodies may include, but may not be limited to, Association for Cooperative Operations Research and Development (ACORD).

The claim processing device 102 may process the insurance claims or a treatment cost payable to a user by an insurance company or a TPA, or under a self-funded or a sponsored medical scheme. By way of an example, the claim processing device 102 may receive a user request via an email from the input computing system 104 for processing of the insurance claims. To this end, the claim processing device 102 may be communicatively coupled to the input computing system 104 via a communication network 108. The claim processing device 102 may further receive a digital data matrix from the data repository 106. To this end, the claim processing device 102 may be communicatively coupled to the data repository 106 via the communication network 108. The communication network 108 may be a wired or a wireless network and the examples may include, but are not limited to the Internet, Wireless Local Area Network (WLAN), Wi-Fi, Long Term Evolution (LTE), Worldwide Interoperability for Microwave Access (WiMAX), and General Packet Radio Service (GPRS).

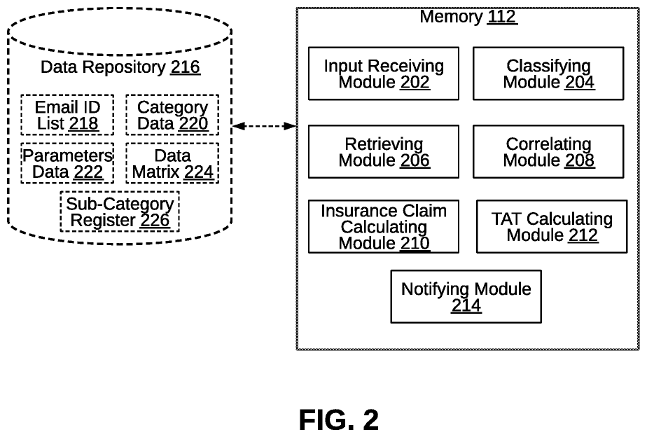

As will be described in greater detail in conjunction with FIG. 2 to FIG. 5, in order to process an insurance claim or a treatment cost payable to a user by an insurance company or a TPA, or under a self-funded or a sponsored medical scheme, the claim processing device 102 may receive a user identity and a user request for awarding of insurance claims payable by the insurance company. The claim processing device 102 may further classify the user request in one of a plurality of pre-defined categories. The claim processing device 102 may further correlate the user request with a digital data matrix. The digital data matrix may be stored in the data repository 106. The claim processing device 102 may further retrieve one or more parameters associated with the user identity. The one or more parameters associated with the user identity may be stored in the data repository 106. The claim processing device 102 may further determine an insurance claim amount payable to the user by the insurance company, based on the correlation with the data matrix and the one or more parameters associated with the user identity.

In order to perform the above discussed functionalities, the claim processing device 102 may include a processor 110 and a memory 112. The memory 112 may store instructions that, when executed by the processor 110, may cause the processor 110 to determine an insurance claim amount payable to the user by the insurance company based on the correlation with the data matrix and the one or more parameters associated with the user identity, and an insurance policy, as discussed in greater detail in FIG. 2 to FIG. 5. The memory 112 may be a non-volatile memory or a volatile memory. Examples of non-volatile memory, may include, but are not limited to a flash memory, a Read Only Memory (ROM), a Programmable ROM (PROM), Erasable PROM (EPROM), and Electrically EPROM (EEPROM) memory. Examples of volatile memory may include, but are not limited to Dynamic Random Access Memory (DRAM), and Static Random-Access memory (SRAM). The memory 112 may also store various data (e.g., user identity data, user request data, pre-defined category data, sub-category registers, digital data matrix, parameters data, insurance policy data, notification data, etc.) that may be captured, processed, and/or required by the system 100.

The claim processing device 102 may further include a user interface 114 through which the claim processing device 102 may interact with a user and vice versa. By way of an example, the user interface 114 may be used to display the as determined insurance claim amount. The user interface 114 may further allow a user to input information into the claim processing device 102. In some embodiments, documents uploaded may be viewed or verified for data entry in a split screen window. The documents uploaded for claims may be stored against a transaction along with a reference number. Additionally, options for download document, zoom in and zoom out, and rotating the documents may be provided, via the user interface 114.

The system 100 may interact with one or more external devices 116 over the communication network 108 for sending or receiving various data. Examples of the one or more external devices 116 may include, but are not limited to a remote server, a digital device, or another computing system.

Referring now to FIG. 2, a functional block diagram of the memory 112 within the claim processing device 102 configured to process an insurance claim or a treatment cost payable to a user by an insurance company or a TPA, or under a self-funded or a sponsored medical scheme is illustrated, in accordance with an embodiment. The memory 112 may include one or more modules that may perform various functions so as to process an insurance claim. The memory 112 may include an input receiving module 202, a classifying module 204, a retrieving module 206, a correlating module 208, an insurance claim calculating module 210, a turn-around-time (TAT) calculating module 212, a notifying module 214, and a data repository 216. As will be appreciated by those skilled in the art, all such afore mentioned modules and data repository 202-216 may be represented as a single module or a combination of different modules. Moreover, as will be appreciated by those skilled in the art, each of the modules and data repository 202-216 may reside, in whole or in parts, on one device or multiple devices in communication with each other.

In some embodiments, the input receiving module 202 may receive a user identity and a user request for awarding of insurance claims payable by the insurance company. In some embodiments, the user request may be received via an email. Accordingly, the input receiving module 202 may be communicatively coupled to an input computing system 104 (shown in FIG. 1) configured to receive an email from a user claiming an insurance claim payment payable by the insurance company. The input receiving module 202 may retrieve a user identity from the data repository 216. In some embodiments, the user identity may be an insurance policy number, an insurance identification number, a unique health insurance identification number (UHID), a certificate of insurance (COI) or a unique identification number assigned to a user. It may be noted that the data repository 216 may include a list of email IDs associated with a multiple users, along with the user identity belonging to each corresponding user. By way of an example, the list of email IDs may be procured from one or more insurance companies. It may be understood, that an insurance company may maintain a list of email IDs of the users who have subscribed to one or more insurance policies provided of that insurance company.

In some embodiments, a pre-authorization request may be received through the email. Upon receiving the email, an inward number may be assigned to the email, and an auto-acknowledgment may be sent to the insurance company. It may be understood that the insurance company may be responsible for paying the insurance claim to a user. An email request with attachment may be assigned to an inward user. In some embodiments, an email server scheduler may be created which may run periodically (for example, every 3 minutes) to identify and fetch incremental emails received in a server. A unique work item number may be generated against the email, and an email body, a subject, a sender information, a date, and a time associated with the email may be stored in the data repository 216. In some embodiments, one or more reimbursement claims requests may be scanned, and a work item number may be assigned to each of the one or more reimbursement claims. An inward request along with an attachment may be assigned to a reimbursement inward user, and an inward number may be generated.

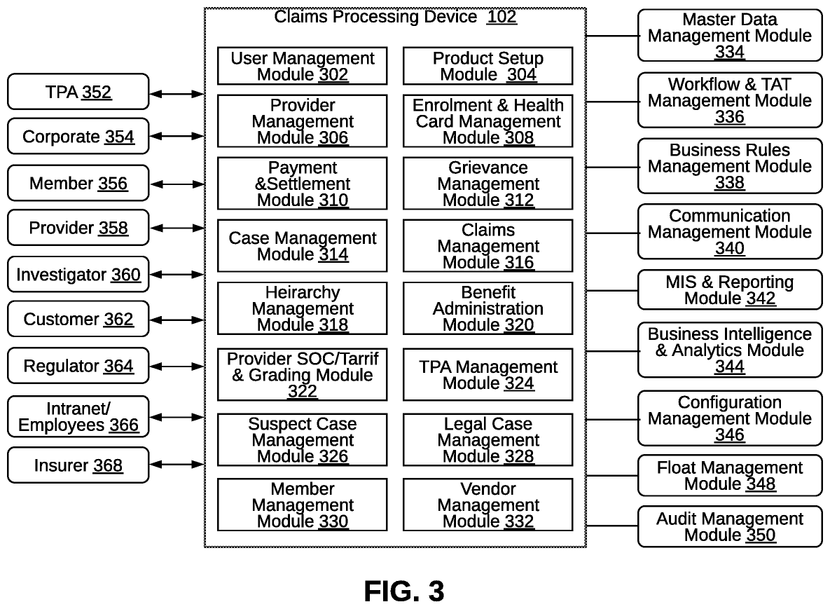

In some embodiments, the input receiving module 202 may lock a work item, so that at a time only one user may be able to process a claim request, in order to avoid duplication of work. By way of an example, the locking of the work item functionality may be achieved through a maintain flag, using a user ID, or a case ID, or a work item ID, or an investigation ID. In some embodiments, based on the email received, a provider may be tagged directly to the claims requested. The provider email ID may be captured in a provider management module (as explained with FIG. 3). In some cases when the email is received from same email ID, the email may be validated and verified. It may be understood that the provider may be a hospital, a clinic or a physician/doctor providing medical service to a patient. In other applications, the provider may be an automobile service/repair center. The data repository 216 may store various data, including email ID list 218, category data 220, parameters data 222, a digital data matrix 224, and one or more sub-category registers 226. As mentioned above, the email ID list 218 may be retrieved from the list of email IDs procured from one or more insurance companies. The category data 220 may include one or more categories for which a user may be claiming the insurance claims. By way of an example, the plurality of pre-defined categories may include a health insurance category and an automobile insurance category.

The classifying module 204 may classify the user request for claim processing in one of the plurality of pre-defined categories, for example, the health insurance category and the automobile insurance category. In other words, the claim processing device 102 may have the capability to process multiple types of the insurance policies including, but not limited to, the health insurance policy and the automobile insurance policy. In some embodiments, the classifying module 204 may further classify the user request in one of one or more sub-categories. The one or more sub-categories may be retrieved from one or more sub-category registers 226 stored in the data repository 216. By way of an example, the one or more sub-category registers 226 may include an International Classification of Diseases (ICD) version 10, a Procedure Coding System (PCS) code, a Current Procedures Terminology (CPT) code, a Systematized Nomenclature of Medicine—Clinical Terms (SNOWMED CT) code and a list of make, model, and variant of automobiles and automobile spare parts.

The retrieving module 206 may retrieve the one or more sub-categories from the one or more sub-category registers 226 stored in the data repository 216. The retrieving module 206 may further retrieve one or more parameters associated with the user identity. By way of an example, for the health insurance category, the one or more parameters associated with the user identity may include age of the user, medical history of the user, and identity of family members related to the user. Similarly, for the automobile insurance category, the one or more parameters associated with the user identity may include a model of the automobile, a make of the automobile, a regional transport office (RTO) associated with the automobile, age, and mileage of the automobile. The one or more parameters associated with the user identity may be stored as parameters data 222 in the data repository 216.

The correlating module 208 may correlate the user request with the digital data matrix 224. The digital data matrix 224 may be stored in the data repository 216. In some embodiments, the digital data matrix 224 may include one or more amounts of insurance claims associated with each of the plurality of pre-defined categories, as defined by each of the insurance companies. Additionally or alternatively, the digital data matrix 224 may include hospital tariffs, as defined by each of the insurance companies, for one or more hospitals registered with each of the insurance companies. In some embodiments, the digital data matrix 224 may include repair costs for various faults associated with each model and make of automobiles in the list of make and model of automobiles, as defined by each of the multiple insurance companies. Additionally or alternatively, the digital data matrix 224 may include repair costs for various faults associated with each model and make of automobiles, as specified by each of multiple service centers. In other words, the digital data matrix 224 may include amounts of insurance claims associated with each of health insurance and automobile insurance, etc., as payable by each of one or more insurance companies.

In some embodiments, the digital data matrix 224 may further include one or more sub-categories associated with the one or more sub-category registers 226. As mentioned above, the one or more sub-category registers 226 may include the ICD 10, the PCS code, the CPT code, the SNOWMED CT code, and a list of make, model, and variant of automobiles and automobile spare parts. By way of an example, each of the ICD 10, the PCS code, and the CPT code may include a list and classification of diseases and related health problems. In such embodiments, the digital data matrix 224 may include costs associated with treatment of each of the diseases and related health problems, as defined by each of the insurance companies. For example, the digital data matrix 224 may include hospital tariffs and schedule of charges (SoC) associated with treatment of each of the diseases and related health problems included in each of the ICD 10, the PCS code, and the CPT code, as defined by each of the insurance companies. Accordingly, the correlating module 208 may correlate the user request with the one or more sub-categories. By way of an example, the correlating module 208 may correlate the user request with the digital data matrix 224 to identify a health condition (for e.g. an ailment) based on the ICD 10 associated with a user.

The insurance claim calculating module 210 may receive the one or more parameters associated with the user identity from the data repository 216. The insurance claim calculating module 210 may further receive the correlation of the user identity with the digital data matrix 224 from the correlating module 208. The insurance claim calculating module 210 may determine an insurance claim amount payable to the user by the insurance company, based on the correlation with the digital data matrix 224 and the one or more parameters associated with the user identity. By way of an example, the insurance claim calculating module 210 may determine an insurance claim amount payable to the user by the insurance company of 1,50,000 Indian National Rupee (INR) for a user request relating to a user aged 40 years and having suffered expenses due to a health condition of Coronary Heart Disease (CHD). In this example, the user may have undergone a Coronary Artery Bypass Grafting (CABG) procedure which may include Operation Theatre (OT) expenses, surgeon's fee, anaesthetist's fee, Intensive Care Unit (ICU) and hospital room charges, nursing charges, and medicine charges. These expenses may be charged item-wise or as a package. In some embodiments the insurance claim calculating module 210 may determine the insurance claim amount using an artificial intelligence (AI) model. The AI model may be trained on the historical data about the insurance claim amount paid to a user under various different circumstances. By way of an example, the AI model may be a Convolutional Neural Network (CNN) model.

In some embodiments, the insurance claim calculating module 210 may apply one or more rules to the one or more parameters associated with the user identity, for calculating the insurance claims amount. By way of an example, according to the one or more rules, weights may be applied to the one or more parameters. In some embodiments, the weights may be assigned by a concerned party (for example, an insurance company). It may be understood that for health insurance, different weights may be assigned to different age groups, during calculating the insurance claims amount. For example, younger age groups may be assigned different weightage than the older age groups, as the insurance claims amount payable to the younger users may be lower than that payable to the older users, and as the insurance premium paid by the older users may be higher than that paid by the younger users. These rule and weights may be stored in the data repository 216 for future usage.

The TAT calculating module 212 calculate a turn-around time (TAT) for processing the insurance claim amount payable to the user by the insurance company. In some embodiments, the TAT calculating module 212 may calculate the TAT based on historical data associated with the insurance company. By way of an example, the historical data associated with the insurance company may include time taken by that insurance company to process the insurance claims and pay the claims amount to the user, in previous cases. The TAT may be configured at each process step involved in the claim processing. The actual time taken may be tracked against a benchmark TAT configured. This may help in improving response time of claim adjudication by the insurance companies, from time to time

The notifying module 214 may generate a notification to at least one of the user and the insurance company, based on the determined insurance claim amount payable to the user by the insurance company. For example, the notifying module 214 may generate the notification via a display device, such as a mobile phone screen. Alternately, the notifying module 214 may send a notification to a user device (for example, a mobile phone) via an email or a text message. In some embodiments, the notifying module 214 may periodically send notifications regarding status of the processing of the claims, as well as TAT associated with the processing of the claims. In some embodiments, the customer may be sent a link (for example, via an SMS) to track the claim status. On clicking the link, a web page may open with tracking records with start point and end point of the process. The current claim stage may be shown to the user along with the status.

Referring now to FIG. 3, a functional block diagram of a system 300, for processing an insurance claim or a treatment cost payable to a user by an insurance company or a third party administrator (TPA), or under a self-funded or a sponsored medical scheme, is illustrated in accordance with an embodiment. In some embodiments, the system 300 may include a claim processing device 102 and a plurality of portals for respective stakeholders to log in to system/application and access various functionalities, as applicable, for processing of the insurance claims. By way of an example, the plurality of portals may include a TPA portal 352, a corporate portal 354, a member portal 356, a provider portal 358, an investigator portal 360, a customer portal 362, a regulator portal 364, an Intranet users or employees portal 366, and an insurer portal 368. The claim processing device 102 may include one or more modules for processing of the insurance claims. In some embodiments, the system 300 may further include one or more peripheral modules associated with the processing of the insurance claims.

In some embodiments, the claim processing device 102 may include a user management module 302, a product setup module 304, a provider management module 306, an enrolment and health card management module 308, a payment and settlement module 310, a grievance management module 312, a case management module 314, a claims management module 316, a hierarchy management module 318, a benefit administration module 320, a provider SoC or tariff management module 322, a TPA management module 324, a suspect case management module 326, a legal case management module 328, a member management module 330, and a vendor management module 332. As will be appreciated, the aforementioned modules 302-332 may be represented as a single module or a combination of different modules. Moreover, as will be appreciated, each of the modules 302-332 may reside, in whole or in parts, on one device or multiple devices in communication with each other. The peripheral modules may include a master data management module 334, a workflow and TAT management module 336, a business rules management module 338, a communication management module 340, a MIS and reporting module 342, a business intelligence and analytics module 344, a configurations management module 346, a float management module 348, and audit management module 350.

The user management module 302 may create and manage new users, organizations and office hierarchies. For example, the user management module 302 may map users to offices to define the hierarchies. The user management module 302 may further assign user privileges for pre-defined user roles. In some cases, the user management module 302 may block or unblock user access. The user access may be blocked or unblocked manually or automatically. In some embodiments, a role-wise and user-wise financial limit (for example, for approval or rejection) may be configured in front end through the user management module 302. The role-wise and user-wise approval and rejection limit may be maintained, and may be validated during claim processing. If a user does not have authority, the system may automatically recommend the case for appropriate authority users for further processing. In some embodiments, predefined privileges may be assigned to roles based on nature of work performed by users. Along with primary role, secondary roles may be assigned to the users. Predefined roles may be created, and set of functionalities may be assigned to each role. One or more roles may be assigned to a user, who may switch the role to use defined functionalities of each roles in a log-in. In some embodiments, user wise activity may be recorded and shown to a respective user. For every user, action logs may be maintained, and these logs may be filtered according to user login and date of login. The records may be shown in activity grids.

In some embodiments, a Lightweight Directory Access Protocol (LDAP) authentication may be provided for a single sign in. An active directory may be integrated to validate internal users with internal system password. In some embodiments, the bulk or Individual communications may be integrated with a mobile messaging application, for example, “WhatsApp” for business. In some embodiments, a chatbot, for example, a “Microsoft” chatbot API may be implemented with predefined configuration based on industry scenarios.

In some embodiments, a user may opt for either a last-in, first-out (LIFO) or a first-in, first-out (FIFO) arrangement of transactions in all the grids, based on date and time. Further, the user may have an option to set default number of transactions to be seen in each page (for example, 5, 10, 25, 50, or 100 transactions). Each grid columns may have filters. In some embodiments, claims of same policy may be shown to a user. For example, last 100 claims under the policy may be filtered and shown for viewing. In some embodiments, Google Distance API may be used to calculate distance between insured resident and hospital location, also Google heat map service may be used to identify geographic location. In some embodiments, Data Mart, and data cubes may be created to keep data in required structure, and which may be used to show counts and analysis.

The product setup module 304 may set up one or more products and schemes. For example, the one or more set up products and schemes may relate to retail, group, mass, and self-funded products and schemes. The product setup module 304 may further set up hospital plans. In some embodiments, the product setup module 304 may configure admission (for example, in hospital) types and lines of treatment available with the hospital under each plan. The product setup module 304 may further cater to product configuration of multiple insurances companies either for inhouse claim adjudication or for the TPAs to adjudicate the claims. The product setup module 304 may have work flow for configuring the product and defining eligibility, limits, benefits, discount etc., and create a copy of the product and enable it for editing. A new plan version may be created with a validity period.

The provider management module 306 may allow empanelment of new providers and renew existing providers. The provider management module 306 may further bulk upload backlisted providers and categorize the providers as blacklisted, bulk upload process for empanelment of multiple provider using excel template and excel upload utility, and bulk upload process for de-empanelment of multiple providers using excel template and excel upload utility. The provider management module 306 may further voluntarily terminate providers upon request, and then proceed for de-empanelment. The provider management module 306 may facilitate creation of Preferred Provider Network (PPN) and corresponding PPN specific charges and rates. The provider management module 306 may further provide for grading and scoring the providers using a matrix of defined parameters, and calculations for grading. The provider management module 306 may further enable editing and updating provider details from time to time. The provider management module 306 may handle renewal of provider empanelment. In some examples, the provider management module 306 may further provision for mapping of hospital codes as assigned by Registry of Hospitals in Network of Insurance (ROHINI), a centralized repository created by Insurance Information Bureau, India (IIB).

The enrolment and health card management module 308 may capture information about one or more insurance policies offered by one or more insurance companies, and the members (users) subscribed to the one or more insurance policies. The enrolment and health card management module 308 may further generate UHID for the members. The enrolment and health card management module 308 may further generate commands for creation of the insurance cards issued to the members, and print these cards. The enrolment and health card management module 308 may further issue group health policies, and may allow for addition and deletion of employees (users) and dependents thereof. In some embodiments, a user (for example, an agent, a broker, a HR manager, a sales manager, or a corporate manager) may upload email IDs of these users against a policy number. These records and the claims may be recorded and sent via email (for example, via carbon copy (CC)). In some embodiments, claim details with status and communication logs and intimation web service is exposed to Microsoft Dynamics customer relationship management (MSCRM) to provide required information to a customer. A web API for intimation may be created, and a dynamic may be created to extract communication log and claims details with status.

The payment and settlement module 310 may provide for individual or bulk payments towards multiple claims, and generation of batch reports for associated banks. The payment and settlement module 310 may further prepare settlement letters and settlement reports for the claims payments already made. Accordingly, the payment and settlement module 310 may generate bank transaction reference the for payments made against claims, and may further provide for payment reconciliation. In some embodiments, MIS & reporting module 342 may enable the user to customize and download required report (custom report) from the front end. All fields may be exposed in the front end for the user to select required column data. Based on the user selection, data may be extracted from a replica database in “Microsoft Excel” format. In some embodiments, based on defined frequency and recipient configured, data may be provided in a predefined “Microsoft Excel” template report (standard report).

The grievance management module 312 may receive and register grievance from a customer (i.e. a user subscribed to an insurance policy), or an insurance company, or a TPA. The grievance management module 312 may further configure escalation matrix for the grievances received, automatically escalate the grievance cases based on the escalation matrix, and track and update status of grievance redressal. The grievance management module 312 may integrate the receiving and redressing of grievance with an insurance company's existing customer relation management (CRM) portals.

The case management module 314 may determine a cost range for expenses associated with a medical treatment type viz., medical or surgical. The expenses may be based on LOS (length of stay) in hospital for treatment, and the expenses may include costs associated to the treatment (for example, operation theatre, surgeon or physician charges, cost of medicines etc.). In other examples, the expenses may include costs associated to repair of an automobile. The case management module 314 may further flag claims falling outside the range set. In some embodiments, the case management module 314 may further configure medical and surgical protocols prescribed for each of one or more medical conditions, so as to highlight discrepancies. The case management module 314 may further configure drug and implant costs. In some embodiments, VIP policies and member flags may be maintained, and death claim flagging option may be provided to a user. Based on these flags, a priority flag may be updated for immediate action.

The claims management module 316 may manage pre-authorization and enhancement of claims award (for example, claims awarded by way of cashless transaction or reimbursement). A unique claim number may be generated. The claims management module 316 may further design workflow for the purpose of pre-authorization, claim submission, and approval. By way of an example, the claims management module 316 may tag claims with ICD 10 codes, and calculate claim liability, upon generating a claim worksheet. In some embodiments, bulk Out Patient Department (OPD) claims may be processed using “Microsoft Excel”. “Microsoft Excel” data may be validated, and upon successful validation, records may be picked through the schedular. The claims management module 316 may validate details passed for the claim asynchronously based on the product, members, and provider configuration. The claims management module 316 may provide for bulk letter generation with date range to help LCU team for dispatch of physical letters, and provide for data exchange between the TPAs and insurance companies using real time API calls, schedulers and offline process with bulk excel upload process.

The hierarchy management module 318 may create and manage new users, organizations and office hierarchies, assign user privileges for user roles defined, and map users to offices. The benefit administration module 320 may configure inclusions, and exclusions in the ICD 10 or PCS code or CPT code or SNOWMED terms, under each plan of the insurance policy. The benefit administration module 320 may upload policy wording, exclusions, benefit chart, etc. under each plan, on a portal. In some embodiments, the benefit administration module 320 may configure limits, sub-limits for room-rent, Intensive Care Unit (ICU), Operation Theatre (OT), medicines, etc. under each plan at a member level, or a family level or a policy level, for medical or health relates insurance claims. Add-on covers may be configured under each plan. The provider SoC or tariff management module 322 may create provider specific tariffs and schedule of charges for various services offered in line with Insurance Regulatory and Development Authority of India (IRDAI) hospital billing regulations. The provider SoC and tariff management module 322 may further configure internal benchmarking for provider SoC and tariffs across different grades of providers and geographies, and configure and maintain PPN rates.

The provider SoC or tariff management module 322 may validate active latest versions of SoC and discounts configured for the attached provider. Accordingly, the provider SoC or tariff management module 322 may automatically deduct discounts, and any amount which may be more than the defined SoC. In some embodiments, one or more versions of SoC and discount (for example, common SoC templates and provider specific templates) may be captured in an IRDA format i.e. format in which level 1, level 2, level 3 services may be coded against various services offered by the provider. A billing structure may be created in the same format to validate SoC and discount at the time of claim processing.

The TPA management module 324 may configure insurance companies or TPAs, capture office details, single point of contact (SPOC) details of insurance companies or TPAs, capture details of TPAs fees and maintain memorandum of understanding (MoU) (for example, between the hospitals and insurance companies or TPAs), and configure frequency of invoicing by the TPAs. The suspect case management module 326 may automatically flag claims for investigation, based on rules, assign suspect cases to internal Investigation team, or to external agencies. The suspect case management module 326 may further capture details along with audio visual evidence, for example, using a mobile application, and flag suspect entities viz. a hospital, and insured member, a medical staff, etc. The suspect case management module 326 may further enable external investigation agencies or investigators to submit investigation details through a mobile application, and enable to upload multimedia files. In some embodiments, post fact cases may be audited to identify operation gaps, issues, mistakes, and remarks with financial loss, and recorded against each claim transaction. The entire claim details may be shown to a user in read only format, and auditor remarks may be captured in each page. In other words, entire summary of audit remarks and financial loss values may be stored against each user, and transaction.

The legal case management module 328 may register legal and assign to empaneled advocates, map legal case to appropriate claims, update status of case after each hearing, and re-open claims and process them, based on verdict from the court. The member management module 330 and the vendor management module 332 may manage the claim payments to the members (i.e. users) subscribed to the one or more insurance policies, payable by the insurance companies (i.e. vendors). The member management module 330 may further provide for maker checker work flow process for enrollment, maker checker work flow process for endorsement, define Report Definition Language Client Side (RDLC) template for card printing using configuration, and provide for bulk card printing using the RDLC. The member management module 330 may further bulk process handling retail and corporate enrollment and endorsement process. In some embodiments, one or more claims for which member insurance policy records are not yet generated, can be processed without insurance policy details through DNF (Data Not Found) work flow process. The initial pre-authorization approval may be modified so as to regularize process again after tagging member and actual insurance policy details to the claim. The DNF flow and validations may be added to handle this scenario.

The master data management module 334 may maintain and update master data, maintain audit trail of master data changes, and bulk upload master data values from “Microsoft Excel” sheets. The master data management module 334 may further manage effective date and expiry date for master data values. The master data management system 334 may receive one or more of, an implant, a medical surgical protocol, and a drug master configuration. These may then be validated during claim processing. Further, master and range values maintained and validated with transaction fields and case management rules may be logged against claims, and flagged with flags MET/NON MET, etc.

The workflow and TAT management module 336 may configure TAT for claim processing from front end for all statuses in the system. The workflow and TAT management module 336 may further perform color coding against defined TAT, for a user to act based on color changes. Based on the color configured for a predefined time, a user may be alerted with a color flag against various transactions. An actual time taken by the user may be maintained for future performance evaluations and analysis of the user.

The business rules management module 338 may configure business rules for STP, suspect or fraud claims. It may be noted that the rules may be run either at pre-authorization level or claims level. The business rules management module 338 may further define rules to run on historic claims or fresh claims, and activate or de-activate rules, as required. The business rules management system 338 may allow a user to create rules for various fields (for example, member, policy, claim, provider fields) captured for claim processing. One or more evaluation and execution rules may be created from front end. It may be noted that the evaluation rules may be informative rules indicating YES/NO results and execution rules may be for initiating a process for taking one or more actions (for example, approve/reject/non register/recommend) when the rule is met. The field names, conditions, master field values may be provided in the front end so as to create business rules. These conditions may be stored and executed on defined triggered points. One or more transaction fields may be validated against rules configured, and all the rules may be logged against claim with one or more flags, such as MET/NON MET. The rules that are met/non-met may be shown to the claims processing system as a list of rules/validations run for a specific claim.

The communication management module 340 may configure message content using templates, or modes of communication, or trigger for the communication. The communication management module 340 may further broadcast messages or instructions to all providers at one-go, and seek acknowledgement of the message being read from the providers. The communication management system 340 may provide a feedback of the member after getting discharged from hospital. A feedback link may be sent along with an approval communication to the customer through an email or SMS. Upon clicking this link, a web page may open with predefined questions relating to the provider service. Further, the customer may rate the provider service (for example, from 1-5). The feedback of each customer may be maintained in the system for provider analysis and recommendations. In some embodiments, a count and details of all transactions of all modules may be shown to a supervisor in according to status, so as to track pending and work under various stages. One or more analytical data tables may be maintained, to keep count and reference numbers of all transactions associated with various stages, status, and users.

The MIS and reporting module 342 may trigger generating of various data for management reporting, operational and transactional reporting, and statutory/compliance reporting from the application. Summary reports may be made available pictorially on dashboards. The reports may be viewed on a screen within the application or may be downloaded in PDF or Microsoft Excel format. The reports and dashboards may be made available to users based on access rights given to the users. The reports may be automatically generated in a batch, and the reports may be sent to respective users. It may be understood that the reports may be generated by users, as and when required.

The business intelligence and analytics module 344 may effectively maintain data so as to address operational, transactional, member, provider, policy related information. The business intelligence and analytics module 344 may support analytics and visualization, so that users may extract information easily by applying insights towards addressing risks, fraud and abuse and forecast future events. The business intelligence and analytics module 344 may help in providing better understanding of patterns in patients and provider behavior. This module may further enable insurance companies in managing claim expenses and quicker settlement of claims and appropriate premium pricing and negotiation with providers.

The configuration management module 346 may provide for configuration of templates for various communications, for example, by way of SMS, E-mail, Whatsapp, etc. This module may handle configuration of workflow, escalation matrix, business rules, and validations configuration.

The float management module 348 may maintain floats for TPAs and automobile garages. By way of an example, the float management module may generate float statement, and workflow for submission or approval of the float statements. The float management module may further make full or partial payment against the float statements. The TPAs and automobile garages may be allowed to view and track status of payments or seek clarifications against float statement submitted.

The audit management module 350 may create and assign requests for auditing of providers, processes, and claims, and seamlessly integrate with a mobile application (for example, “Evidens”) for auditing. An auditing user may be able to capture and update audit findings, and view audit findings submitted using the mobile application.

In some embodiments, the member portal 356 and the corporate portal 354 may generate role-based access to corporate health insurance customers. The roles may include a corporate administrator, a corporate human resource (HR) member and corporate members. The member portal 356 and the corporate portal 356 may further configure specific privileges for each corporate. The member portal 356 and the corporate portal 354 may further perform various functionalities including claim intimation, enrollment of dependents, addition of members mid-term, downloading electronic health (eHealth) card, responding to queries, etc. In some embodiments, a provider through the provider portal 358 may raise requests for pre-authorization of awarding of insurance claims and for enhancement of the insurance claims award. The provider portal 358 may also allow checking of the status of the processing of the claims. The provider portal 358 may further provide for viewing and responding to queries raised by a claims adjudicator (for example, through a portal), and submitting replies to the queries raised and tracking the final status of claims on the portal.

In some embodiments, an offline document management system (DMS) may be provided to decrease dependency on the online DMS. A local storage may be created where the documents may be stored for fifteen days, after which, the documents may be pushed to the DMS through a schedular. During claim processing, the documents may be downloaded multiple times by different users.

In some embodiments, Content Management Interoperability Services (CIMS) may be integrated through an application program interface (API) to fetch prices of medicines based on brand and drug details, which may then be are shown to the claim processing device 102.

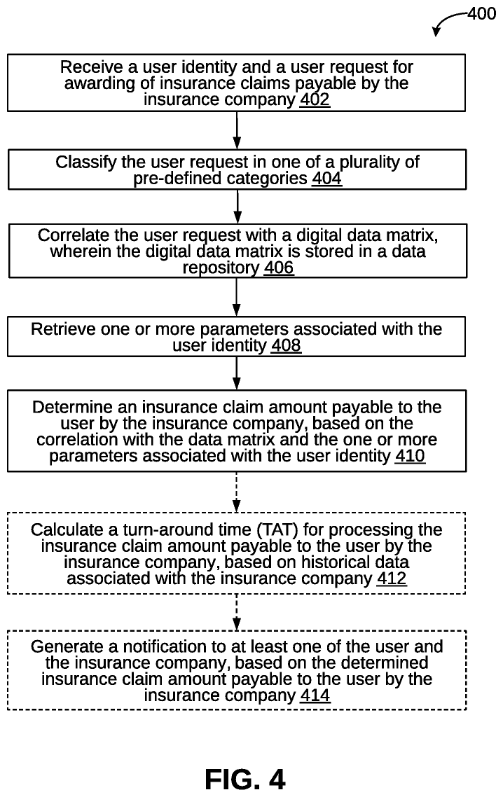

Referring now to FIG. 4, a flowchart 400 of a method of processing an insurance claim or a treatment cost payable to a user by an insurance company or a TPA, or under a self-funded or a sponsored medical scheme is illustrated, in accordance with an embodiment. It may be noted that the various steps of the method, as described herein, may be performed in compliance with one or more standards set by one or more global standards-setting bodies like ACORD. In some embodiments, the method 400 may be performed by the claim processing device 102 (of system 100, as shown in FIG. 1). At step 402, a user identity and a user request may be received for awarding of insurance claims payable by the insurance company. At step 404, the user request may be classified in one of a plurality of pre-defined categories. At step 406, the user request may be correlated with a digital data matrix. At step 408, one or more parameters associated with the user identity may be retrieved. At step 410, an insurance claim amount payable to the user by the insurance company may be determined, based on the correlation with the data matrix and the one or more parameters associated with the user identity. Additionally, at step 412, a turn-around time (TAT) may be calculated for processing the insurance claim amount payable to the user by the insurance company, based on historical data associated with the insurance company. At step 414, a notification may be generated to at least one of the user and the insurance company, based on the determined insurance claim amount payable to the user by the insurance company.

At step 402, the user identity and the user request may be received for awarding of insurance claims payable by the insurance company. In some embodiments the user request may be received via an e-mail from the input computing system 104 (shown in FIG. 1) configured to receive an email from a user claiming an insurance claim. The user identity may be retrieved from the data repository 216 (as shown in FIG. 2). The data repository 216 may include a list of email IDs (for example, procured from one or more insurance companies) associated with a multiple users.

At step 404, the user request may be classified in one of the plurality of the pre-defined categories. By way of an example, the plurality of pre-defined categories may include a health insurance category, and an automobile insurance category. In some embodiments, the user request further may be classified in one of one or more sub-categories. The one or more sub-categories may be retrieved from one or more sub-category registers 226 stored in the data repository 216. By way of an example, the one or more sub-category registers 226 may include an ICD 10, a PCS code, a CPT code, a SNOWMED CT code and a list of make and model of automobiles.

At step 406, the user request may be correlated with the digital data matrix 224. The digital data matrix 224 may be stored in the data repository 216. The data matrix 224 may include at least one of hospital tariffs, a SoC, and automobile repair or maintenance costs. The correlating of the user request with the digital data matrix 224 is already explained in conjunction with FIG. 2. At step 408, the one or more parameters associated with the user identity may be retrieved. The one or more parameters associated with the user identity may include age of the user, medical history of the user, family members identity of the user, model of the automobile, make of the automobile, RTO associated with the automobile, etc. The one or more parameters associated with the user identity may be stored in the data repository 216.

At step 410, an insurance claim amount payable to the user by the insurance company may be determined, based on the correlation with the data matrix and the one or more parameters associated with the user identity. At step 412, a turn-around time (TAT) may be calculated for processing the insurance claim amount payable to the user by the insurance company, based on historical data associated with the insurance company. At step 414, a notification may be generated to at least one of the user and the insurance company, based on the determined insurance claim amount payable to the user by the insurance company.

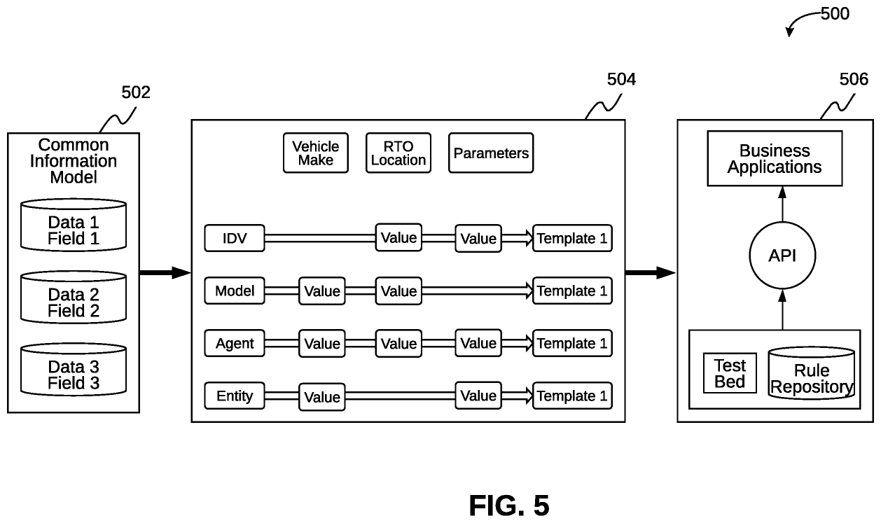

It may be noted that insurance companies may currently use multiple transaction systems for generating quotations, policy issuance and endorsements. Underwriting rules and premium rating logic may be maintained in respective transaction systems. However, implementing changes to business rules may be labour intensive, cumbersome, time-consuming, and expensive, as same changes may have to be implemented across all transactions system. In view of this, a centralized business rule engine may be provided for a centralized view of the underwriting rules and pricing guidelines for an underwriting or an operations team, which may enable easy configuration and management of business rules. A process of providing a centralized view of underwriting rules and pricing guidelines is further explained in conjunction with FIG. 5.

Referring now to FIG. 5, a process 500 of providing a centralized view of underwriting rules and pricing guidelines for processing an insurance claim or a treatment cost payable to a user by an insurance company or a third party administrator (TPA), or under a self-funded or a sponsored medical scheme is illustrated, in accordance with an embodiment. At step 502, master data and field values from across multiple transaction systems may be standardized and prepared for configuration. At step 504, a rule template or an authority matrix may be created for each user role, or user, or entity. The rule template may be “Microsoft Excel” based. The rule template may automatically pre-fill values and create place holders for configurations of values. At step 506, APIs may provide execution of the configured business rules. In some embodiments, transaction systems calling a business rule engine may perform a one-time integration with a centralized business rule engine for the underwriting or operations team which may be impacting the TAT for approval to quotations or policies.

As will be appreciated by those skilled in the art, the techniques described in the various embodiments discussed above pertain to automatic processing of insurance claims payable by an insurance company to a user (insurance policy buyer). The techniques provide for quick and accurate processing of insurance claims. Moreover, the techniques can be implemented over a cloud network, for mobile application. The techniques are further able to identify fraud and suspicious insurance claims. Further, the techniques are capable of mapping acceptable medical protocols, drugs, implants against diagnosis to identify outlier cases. The techniques further provide for quick approvals from insurance companies, faster settlement of cashless claims, and real-time and periodic update on the claims (for example, via a mobile application). By straight-through processing of preauthorized and reimbursement claims, the turn-around time (TAT) for approval of the claims is significantly reduced.

It is intended that the disclosure and examples be considered as exemplary only, with a true scope and spirit of disclosed embodiments being indicated by the following claims.

Glossary of systems, devices and modules

| | | |
|---|---|---|
| 1 | 100 | System for processing an insurance claim or a treatment cost |
| | | payable to a user by an insurance company or a TPA, or |
| | | under a self-funded or a sponsored medical scheme |
| 2 | 102 | Claim processing device |
| 3 | 104 | Input computing system |
| 4 | 106 | Data repository |
| 5 | 108 | Communication network |
| 6 | 110 | Processor |
| 7 | 112 | Memory |
| 8 | 114 | User interface |
| 9 | 116 | External devices |
| 10 | 202 | Input receiving module |
| 11 | 204 | Classifying module |
| 12 | 206 | Retrieving module |
| 13 | 208 | Correlating module |
| 14 | 210 | Insurance claim calculating module |
| 15 | 212 | Turn-around-time (TAT) calculating module |
| 16 | 214 | Notifying module |
| 17 | 216 | Data repository |
| 18 | 218 | Email ID list |
| 19 | 220 | Category data |
| 20 | 222 | Parameters data |
| 21 | 224 | Digital data matrix |
| 22 | 226 | Sub-category registers |
| 23 | 302 | User management module |
| 24 | 304 | Product setup module 304 |
| 25 | 306 | Provider management module 306 |
| 26 | 308 | Enrolment and health card management module |
| 27 | 310 | Payment and settlement module |
| 28 | 312 | Grievance management module |
| 29 | 314 | Case management module |
| 30 | 316 | Claims management module |
| 31 | 318 | Hierarchy management module |
| 32 | 320 | Benefit administration module |
| 33 | 322 | Provider Schedule of Charges (SOC) or tariff management |
| | | module |
| 34 | 324 | Third party administrator (TPA) management module |
| 35 | 326 | Suspect case management module |
| 36 | 328 | Legal case management module |
| 37 | 330 | Member management module |
| 38 | 332 | Vendor management module |
| 39 | 334 | Master data management module |
| 40 | 336 | Workflow and turn-around time (TAT) management |
| | | module |
| 41 | 338 | Business rules management module |
| 42 | 340 | Communication management module |
| 43 | 342 | MIS and reporting module |
| 44 | 344 | Business intelligence and analytics module |
| 45 | 346 | Configurations management module |
| 46 | 348 | Float management module |
| 47 | 350 | Audit management module |
| 48 | 352 | TPA portal |
| 49 | 354 | Corporate portal |
| 50 | 356 | Member portal |
| 51 | 358 | Provider portal |
| 52 | 360 | Investigator portal |
| 53 | 362 | Customer portal |
| 54 | 364 | Regulator portal |
| 55 | 366 | Intranet users or employees portal |
| 56 | 368 | Insurer portal |

What is claimed is:

1. A method of processing an insurance claim payable to a user by an insurance company, the method comprising:
receiving, by a claim processing device, a user identity and a user request for awarding of insurance claims payable by the insurance company;
classifying, by the claim processing device, the user request in one of a plurality of pre-defined categories;
correlating, by the claim processing device, the user request with a digital data matrix, wherein the digital data matrix is stored in a data repository;
retrieving, by the claim processing device, one or more parameters associated with the user identity, wherein the one or more parameters associated with the user identity are stored in the data repository; and
determining, by the claim processing device, an insurance claim amount payable to the user by the insurance company, based on the correlation with the data matrix and the one or more parameters associated with the user identity and an insurance policy.

2. The method as claimed in claim 1, wherein the user request is received via an e-mail, and the user identity is retrieved from the data repository, by mapping an e-mail ID associated with the received email with a list of email IDs stored in the data repository.

3. The method as claimed in claim 1, wherein the plurality of pre-defined categories comprises at least a health insurance category, and an automobile insurance category.

4. The method as claimed in claim 3, wherein the determining an insurance claim amount payable to the user further comprises classifying the user request in one of one or more sub-categories, wherein the one or more sub-categories are retrieved from one or more sub-category registers, and wherein the one or more sub-category registers comprise definition of insurance policy coverage, one or more benefits, limits, and exclusions associated with the insurance policy, and one or more conditions with respect to an International Statistical Classification of Diseases and Related Health Problems (ICD) version 10, a Procedure Coding System (PCS) code, a Current Procedures Terminology (CPT) code, a Systematized Nomenclature of Medicine—Clinical Terms (SNOWMED CT) code and a list of make, model, and variant of automobiles and automobile spare parts.

5. The method as claimed in claim 1 further comprises generating a notification to at least one of the user and the insurance company, based on the determined insurance claim amount payable to the user by the insurance company.

6. The method as claimed in claim 1, wherein the data matrix comprises at least one of hospital tariffs, a schedule of charges (SoC), and automobile repair or maintenance costs.

7. The method as claimed in claim 1, wherein the one or more parameters associated with the user identity comprise age of the user, medical history of the user, family members identity of the user, model of the automobile, make of the automobile, and regional transport office (RTO) associated with the automobile.

8. The method as claimed in claim 1 further comprising calculating a turn-around time (TAT) for processing the insurance claim amount payable to the user by the insurance company, based on historical data associated with the insurance company.

9. A system for processing an insurance claim payable to a user by an insurance company, the system comprising:
a claim processing device, the claim processing device further comprising;
a processor; and
a memory communicatively coupled to the processor, wherein the memory stores processor instructions, which, on execution, causes the processor to:
receive a user identity and a user request for awarding of insurance claims payable by the insurance company;
classify the user request in one of a plurality of pre-defined categories;
correlate the user request with a digital data matrix, wherein the digital data matrix is stored in a data repository;
retrieve one or more parameters associated with the user identity, wherein the one or more parameters associated with the user identity are stored in the data repository; and
determine an insurance claim amount payable to the user by the insurance company based on the correlation with the data matrix and the one or more parameters associated with the user identity, and an insurance policy.

10. The system as claimed in claim 9, wherein the user request is received via an e-mail, and the user identity is retrieved from the data repository, by mapping an e-mail ID associated with the received email with a list of email IDs stored in the data repository.

11. The system as claimed in claim 9, wherein the plurality of pre-defined categories comprises at least a health insurance category, and an automobile insurance category.

12. The system as claimed in 11, wherein the determining an insurance claim amount payable to the user further comprises classifying the user request in one of one or more sub-categories, wherein the one or more sub-categories are retrieved from one or more sub-category registers, and wherein the one or more sub-category registers comprise a definition of insurance policy coverage, one or more benefits, limits, and exclusions associated with the policy coverage, and one or more conditions with respect to an International Statistical Classification of Diseases and Related Health Problems (ICD) version 10, a Procedure Coding System (PCS), a Current Procedures Terminology (CPT) code, a Systematized Nomenclature of Medicine—Clinical Terms (SNOWMED CT) code and a list of make, model, and variant of automobiles, and automobile spare parts.

13. The system as claimed in 9, wherein the processor instructions further cause the processor to generate a notification to at least one of the user and the insurance company, based on the determined insurance claim amount payable to the user by the insurance company, via a display device.

14. The system as claimed in 9, wherein the data matrix comprises at least one of hospital tariffs and automobile repair and maintenance costs, and wherein the one or more parameters associated with the user identity comprise age of the user, medical history of the user, family members identity of the user, model of the automobile, make of the automobile, and regional transport office (RTO) associated with the automobile.

15. The system as claimed in 9, wherein the processor instructions further cause the processor to predict a turn-around time (TAT) for processing the insurance claim amount payable to the user by the insurance company, based on historical data associated with the insurance company.
